(19) United States
(12) Patent Application Publication
(10) Pub. No.: US 2026/0355116 A1
(43) Pub. Date: Nov. 12, 2026

(54) COMPOSITIONS FOR ORAL DELIVERY

(71) Applicant: ELI LILLY AND COMPANY, Indianapolis, IN (US)

(72) Inventors: Aktham ABURUB, Carmel, IN (US); Siyuan HUANG, Indianapolis, IN (US); Jennifer Marie WALKER, Zionsville, IN (US)

(21) Appl. No.: 19/106,042

(22) PCT Filed: Aug. 28, 2023
(86) PCT No.: PCT/US2023/072968

§ 371 Date: Feb. 24, 2025

Related U.S. Application Data
(60) Provisional application No. 63/373,776, filed on Aug. 29, 2022.

(51) Int. Cl. A61K 9/48; A61K 38/26

(52) U.S. Cl. CPC A61K 9/4891; A61K 9/4808; A61K 38/26

(57) ABSTRACT

The present invention relates to a dosage form suitable for oral administration comprising a therapeutic peptide or protein in a sealed capsule having a co-polymer coating.

The present invention is in the field of medicine. More particularly, the present invention relates to a solid pharmaceutical composition comprising a therapeutic peptide or protein which is suitable for oral delivery. More particularly, the present invention relates to a solid pharmaceutical composition in which the therapeutic peptide is an incretin analog or derivative with activity at the glucagon-like peptide (GLP-1) receptor, the glucose-dependent insulinotropic polypeptide (GIP) receptor and/or the glucagon (GCG) receptor. The compositions of the present invention comprise a therapeutic peptide that is a single GIP, GLP-1, or GCG receptor agonist, a dual GIP/GLP-1 or GLP-1/GCG receptor agonist, or a triple GIP/GLP-1/GCG receptor agonist and may be useful in the treatment of type 2 diabetes (T2D), obesity, nonalcoholic fatty liver disease (NAFLD), nonalcoholic steatohepatitis (NASH), cardiovascular disease (CVD) and/or in the prevention of cognitive decline.

### BACKGROUND OF THE INVENTION

Most existing incretin analogue therapies are administered parenterally, but efforts have been focused recently on development of incretin therapies that may be administered orally, which allows the patient to self-administer the therapeutic peptides and may result in improved patient compliance and adherence.

The formulation of a therapeutic peptide or protein into an oral formulation remains challenging and unpredictable, however, due in part to the susceptibility of peptides to the proteolytic and pH conditions that exist in the different portions of the digestive tract. The inefficient transport of biologics, including incretin peptides, across the stomach and/or the intestinal wall, therefore, has been a technology challenge for the oral delivery of therapeutics. Most of the active pharmaceutical ingredient (API) is rapidly degraded or not absorbed, typically resulting in insufficient systemic bioavailability. Large amounts of API, therefore, are required in order to administer an effective therapeutic dose. Most of the costly API is thus wasted, and a large tablet may be difficult for the patient to swallow.

The only presentation for oral administration of a peptide for use in the treatment of type 2 diabetes mellitis (T2DM) is the product containing the GLP-1 receptor agonist semaglutide marketed under the tradename RYBELSUS. This product must be administered in a fasted state, however, and the bioavailability of semaglutide from this product is only about 1%.

Efforts have been made to develop improved compositions and dosage forms. For example, WO2022049310 describes solid oral pharmaceutical compositions stated to provide improved bioavailability.

Nevertheless, there remains a need for new therapies providing greater bioavailability than currently available therapies. There also remains a need for new therapies with greater flexibility in administration, in particular with no requirements regarding administration in either a fed or fasted state.

### BRIEF SUMMARY OF THE INVENTION

The present invention provides compositions suitable for oral administration of proteins or peptides. The compositions are designed to provide greater bioavailability as compared to currently available therapies that are orally administered. The compositions also may be administered to a patient in either a fasted or a fed state.

According to a first aspect of the present invention there is provided a solid oral pharmaceutical dosage form comprising:
a) a core composition comprising a therapeutic protein or peptide;
b) a capsule that contains the core composition and wherein the capsule has a body and a cap;
c) a polymeric seal covering the transition between the capsule cap and body; and
d) an enteric coating that coats the polymeric seal and capsule.

According to a preferred embodiment, the dosage form releases: none of the therapeutic peptide or protein in fluid having pH≤4.5 and releases the majority of the peptide or protein in fluid having pH≥6.0.

According to another preferred embodiment, the dosage form releases: none of the therapeutic peptide or protein for up to 2 hours in fluid having pH between 4.5 and 6.0; and the majority of the peptide or protein in fluid having pH≥6.0.

According to a preferred embodiment, the dosage form releases: none of the therapeutic peptide or protein for up to 2 hours in fluid having pH between 4.5 and 6.0; and the majority of the peptide or protein in fluid having pH≥6.8.

According to another preferred embodiment, the enteric coating comprises a co-polymer comprising at least one polymer selected from the group consisting of methyl acrylate, methyl methacrylate, methacrylic acid and ethyl acrylate.

According to another preferred embodiment, the enteric coating comprises poly[methacrylic acid, ethyl acrylate] wherein the methacrylic acid and ethyl acrylate are present in a ratio of about 1:1.

According to another preferred embodiment, the enteric coating comprises poly[methyl acrylate, methyl methacrylate, methacrylic acid] in a ratio of about 7:3:1.

According to another preferred embodiment, the therapeutic peptide or protein is an analog of peptide tyrosine-tyrosine (PYY) or has agonistic activity at one or more of the glucose-dependent insulinotropic polypeptide (GIP), glucagon-like peptide-1 (GLP-1), glucagon (GCG), amylin and/or calcitonin receptors.

According to another preferred embodiment, the core composition further comprises a permeation enhancer is selected from the group consisting of sodium N-[8-(2-hydroxybenzoyl) amino] caprylate (SNAC), salcaprozate sodium, sodium caprate (C10), or 8-(N-2-hydroxy-5-chlorobenzoyl)-amino-caprylic acid (5-CNAC).

According to another preferred embodiment, the relative bioavailability of the therapeutic peptide or protein following oral administration of the dosage form is greater than 1% of what would be achieved through subcutaneous administration.

According to another preferred embodiment, the bioavailability of the therapeutic peptide or protein on oral administration of the dosage form is not negatively impacted if administered with food.

According to another aspect of the present invention there is provided a solid oral pharmaceutical dosage form comprising:
a) a core composition comprising a therapeutic protein or peptide and C10;
b) a capsule that contains the core composition and wherein the capsule has a body and a cap;
c) a polymeric seal covering the transition between the capsule cap and body; and
d) an enteric coating that coats the polymeric seal and capsule, wherein the coating comprises one or more co-polymers selected from the group consisting of:
   i) poly[methacrylic acid, ethyl acrylate], wherein the methacrylic acid and ethyl acrylate are present in a ratio of about 1:1; and
   ii) poly[methyl acrylate, methyl methacrylate, methacrylic acid], wherein the methyl acrylate, methyl methacrylate and methacrylic acid are present in a ratio of about 7:3:1.

In another aspect, there is provided a solid pharmaceutical dosage form for oral administration comprising: (a) a core composition comprising: (i) 5-25 mg of tirzepatide; and (ii) about 280 mg C10; (b) a capsule that contains the core composition and that has a body and a cap; (c) a polymeric seal over the transition between the capsule cap and body comprising: (i) about 17% HPMC; (ii) about 58% ethanol; and (iii) about 25% water; and (d) a coating that coats the polymeric seal and capsule and that comprises: (i) about 12.9% poly[methacrylic acid, ethyl acrylate], wherein the methacrylic acid and ethyl acrylate are present in a ratio of about 1:1; (ii) about 51.6% poly[methyl acrylate, methyl methacrylate, methacrylic acid], wherein the methyl acrylate, methyl methacrylate and methacrylic acid are present in a ratio of about 7:3:1; (iii) about 2.9% TEC; and (iv) about 32.6% water; and wherein the coating level is about 10 mg/cm².

In another aspect, there is provided a solid pharmaceutical dosage form for oral administration comprising: (a) a core composition comprising: (i) 1-25 mg of a therapeutic peptide or protein; (ii) about 280 mg C10; (iii) about 66 mg MCC; (b) a capsule that contains the core composition and that has a body and a cap; (c) a polymeric seal over the transition between the capsule cap and body comprising: (i) about 17% HPMC; (ii) about 58% ethanol; and (iii) about 25% water; and (d) a coating that coats the polymeric seal and capsule and that comprises: (i) about 12.9% poly[methacrylic acid, ethyl acrylate], wherein the methacrylic acid and ethyl acrylate are present in a ratio of about 1:1; (ii) about 51.6% poly[methyl acrylate, methyl methacrylate, methacrylic acid], wherein the methyl acrylate, methyl methacrylate and methacrylic acid are present in a ratio of about 7:3:1; (iii) about 2.9% TEC; and (iv) about 32.6% water; and wherein the coating level is about 10 mg/cm².

In another aspect, there is provided a solid pharmaceutical dosage form for oral administration comprising: (a) a core composition comprising: (i) 1-25 mg of a therapeutic peptide or protein; (ii) about 280 mg C10; (iii) about 66 mg MCC; (b) a capsule that contains the core composition and that has a body and a cap; (c) a polymeric seal over the transition between the capsule cap and body comprising: (i) about 17% HPMC; (ii) about 58% ethanol; and (iii) about 25% water; and (d) a coating that coats the polymeric seal and capsule and that comprises: (i) about 64.5% poly[methacrylic acid, ethyl acrylate], wherein the methacrylic acid and ethyl acrylate are present in a ratio of about 1:1.; (ii) about 2.9% TEC; and (iii) about 32.6% water; and wherein the coating level is about 7 mg/cm².

According to another aspect of the present invention there is provided a method of treating a disease or condition in a patient in need thereof comprising administering a dosage form as described herein.

According to another aspect of the present invention there is provided a dosage form of the present invention for use in the treatment of a disease or condition in a patient.

According to a preferred embodiment, the dosage form may be administered with or without food. According to a preferred embodiment, the disease or condition is selected from the group consisting of type 2 diabetes mellitus (T2DM), obesity, cardiovascular disease (CVD), non-alcoholic steatohepatitis (NASH), fatty liver disease (FLD), dyslipidemia, metabolic syndrome, cognitive decline, Parkinson's syndrome and Alzheimer's disease.

According to another aspect of the present invention there is provided a process for preparing a dosage form as described herein, comprising:
a) blending all components comprised in the core composition in a suitable blender;
b) weighing an amount of the blend formed in step (a) to be the core composition;
c) compressing the blend weighed in step (b) using a capsule slug mold; d) placing the slug formed in step (c) into the capsule;
e) covering the transition between the capsule cap and body with the polymeric seal; and
f) coating the capsule with the enteric coating.

### BRIEF DESCRIPTION OF THE DRAWINGS

FIG. 1. Illustration of protocol for clinical study designed to evaluate dosage forms of the present invention.

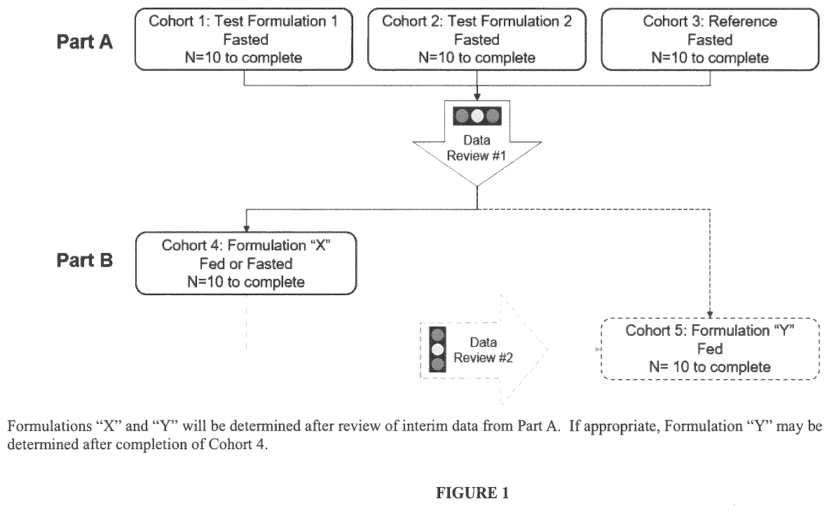

### DETAILED DESCRIPTION OF THE INVENTION

The present invention provides a dosage form for oral administration. The dosage forms described in the present disclosure are composed of a core composition that comprises a therapeutic peptide or protein and that is contained in a capsule that is sealed and coated with an enteric coating.

It has been found that oral dosage forms that release therapeutic peptide or proteins in the middle or distal regions of the intestine provide higher bioavailability of the therapeutic protein or peptide as compared to those that release earlier in the digestive tract, such as the stomach, duodenum or proximal region of small intestine. It has also been found that such targeted release may be achieved through the use of the dosage forms described herein.

Surprisingly, preferred dosage forms described herein are able to achieve such targeted release regardless of whether they are administered to the patient in a fed or fasted state. The potential benefits of the availability of such flexible dosing timing and scheduling include improved adherence and efficacy.

The drug release profiles of dosage forms described herein targeting the middle or distal regions of the intestine are achieved through a combination of the sealed capsules and enteric coatings described herein.

In order to achieve release profiles in the desired regions of the intestine, dosage forms of the present invention preferably release none of the therapeutic peptide or protein in fluid having pH≤4.5. Preferably, the dosage form releases the majority of the peptide or protein in fluid having pH≥6.0. In certain embodiments targeting distal delivery, the dosage form releases the majority of the peptide or protein in fluid having pH≥6.8. In certain preferred embodiments targeting distal delivery, the dosage form releases: none of the therapeutic peptide or protein for up to 4 hours in fluid having pH≤4.5; none of the therapeutic peptide or protein for up to 2 hours in fluid having pH between 4.5 and 6.0; and the majority of the peptide or protein in fluid having pH≥6.8. In certain preferred embodiments targeting distal delivery, the dosage form releases the majority of the peptide or protein within 4 hours in fluid having pH≥6.8.

The desired release profile of dosage forms described herein may be determined with in vitro dissolution testing procedures known in the art, including for example those described in the examples below.

Dosage forms designed to target different regions of the intestine have been described previously. See, e.g., Maroni, Alessandra, et al. In vitro and in vivo evaluation of an oral multiple-unit formulation for colonic delivery of insulin. EUROPEAN JOURNAL OF PHARMACEUTICS AND BIOPHARMACEUTICS 108 (2016): 76-82; Schellekens, R. C. A., et al. Pulsatile drug delivery to ileo-colonic segments by structured incorporation of disintegrants in pH-responsive polymer coatings. JOURNAL OF CONTROLLED RELEASE 132.2 (2008): 91-98; Liu, Fang, et al. Evolution of a physiological pH 6.8 bicarbonate buffer system: application to the dissolution testing of enteric coated products. European Journal of Pharmaceutics and Biopharmaceutics 78.1 (2011): 151-157; Maroni, Alessandra, et al. In vitro and in vivo evaluation of an oral multiple-unit formulation for colonic delivery of insulin. EUROPEAN JOURNAL OF PHARMACEUTICS AND BIOPHARMACEUTICS 108 (2016): 76-82; Schellekens, R. C. A., et al. Pulsatile drug delivery to ileo-colonic segments by structured incorporation of disintegrants in pH-responsive polymer coatings. JOURNAL OF CONTROLLED RELEASE 132.2 (2008): 91-98; Liu, Fang, et al. Evolution of a physiological pH 6.8 bicarbonate buffer system: application to the dissolution testing of enteric coated products. EUROPEAN JOURNAL OF PHARMACEUTICS AND BIOPHARMACEUTICS 78.1 (2011): 151-157.

Enteric coatings that have been found to provide the targeted release characteristics described herein include coatings comprised of one or more polymers or co-polymers. Nonlimiting examples of such polymers or co-polymers include those identified in Table 1 below:

TABLE 1
Polymers or co-polymers for use in enteric coatings.

| | Polymer | |
|---|---|---|
| Chemical Name | Ratio | Example Trade name(s) |
| poly[butyl methacrylate, (2- | 1:2:1 | Eudragit ® E 100, Eudragit ® |
| dimethyaminoethyl) methacrylate, | | E12.5, |
| methyl methacrylate | | Eudragit ® E PO |
| Poly[ethyl acrylate, methyl | 2:1 | Eudragit ® NE 30 D, Eudragit ® |
| methacrylate] | | NE 40 D, Eudragit ® NM 30 D |
| Poly [methacrylic acid, methyl | 1:1 | Eudragit ® L 100, Eudragit ® L |
| methacrylate] | | 12.5, |
| | | Eudragit ® L 12.5 P |
| Poly[methacrylic acid, ethyl acrylate] | 1:1 | Acryl-EZE, Acryl-EZE 93A, |
| | | Acryl-EZE MP, Eudragit ® L30 D- |
| | | 55, Eudragit ® L 100-55, Eastacryl |
| | | 30 D, Kollicoat ® MAE 30 DP, |
| | | Kollicoat ® MAE 100 P |
| Poly[methacrylic acid, methyl | 1:2 | Eudragit ® S 100, Eudragit ® S |
| methacrylate] | | 12.5, Eudragit ® S 12.5 P |
| Poly[methyl acrylate, methyl | 7:3:1 | Eudragit ® FS30 D |
| methacrylate, methacrylic acid] | | |
| Poly[ethyl acrylate, methyl | 1:2:0.2 | Eudragit ® RL 100, Eudragit ® RL |
| methacrylate, trimethylammonioethyl | | PO, Eudragit ® RL 30D, Eudragit ® |
| methacrylate chloride] | | RL 12.5 |
| Poly[ethyl acrylate, methyl | 1:2:0.1 | Eudragit ® RS 100, Eudragit ® RS |
| methacrylate, trimethylammonioethyl | | PO, Eudragit ® RS 30 D, |
| methacrylate chloride] | | Eudragit ® RS 12.5 |
| HPMCAS (H) | NA | |
| Hydroxyl propyl methyl cellulose | NA | |
| phthalate (HPMCP) | | |
| Polyvinyl acetate phthalate (PVAP) | NA | |
| Cellulose acetate trimellitate (CAT) | NA | |
| Cellulose acetate phthalate (CAP) | NA | |

Trade names listed are commercially available products that comprise the polymers or co-polymers indicated, in many cases as aqueous dispersions.

Table 1. Polymers or co-polymers for use in enteric coatings. Trade names listed are commercially available products that comprise the polymers or co-polymers indicated, in many cases as aqueous dispersions.

In certain preferred embodiments, coatings that may be used in dosage forms of the present invention are comprised of one or more of Poly[methacrylic acid, ethyl acrylate] (1:1 ratio) (e.g., Eudragit® L30D-55), Poly[methyl acrylate, methyl methacrylate, methacrylic acid] (7:3:1 ratio) (e.g., Eudragit® FS30D), Poly[methacrylic acid, methyl methacrylate] (1:2 ratio) (e.g., Eudragit® S), Poly[methacrylic acid, methyl methacrylate] (1:1 ratio) (e.g., Eudragit® L) and HPMCAS (H). Particularly preferred co-polymers include Poly[methacrylic acid, ethyl acrylate] (1:1 ratio) (e.g., Eudragit® L30D-55), Poly[methyl acrylate, methyl methacrylate, methacrylic acid] (7:3:1 ratio) (e.g., Eudragit® FS30D) and mixtures thereof.

In certain preferred embodiments, the coating comprises include between 10 and 70% Poly[methyl acrylate, methyl methacrylate, methacrylic acid] (7:3:1 ratio) (e.g., Eudragit® FS30D). In certain preferred embodiments the coating comprises about 51.6% Poly[methyl acrylate, methyl methacrylate, methacrylic acid] (7:3:1 ratio) (e.g., Eudragit® FS30D). In certain preferred embodiments the coating comprises about 53.3% Poly[methyl acrylate, methyl methacrylate, methacrylic acid] (7:3:1 ratio) (e.g., Eudragit® FS30D).

In certain preferred embodiments the coating comprises a combination of Poly[methacrylic acid, ethyl acrylate] (1:1 ratio) (e.g., Eudragit® L30D-55), Poly[methyl acrylate, methyl methacrylate, methacrylic acid] (7:3:1 ratio) (e.g., Eudragit® FS30D). In certain preferred embodiments the coating comprises a combination of about 13.3 percent Poly[methacrylic acid, ethyl acrylate] (1:1 ratio) (e.g., Eudragit® L30D-55) and about 53.3 percent Poly[methyl acrylate, methyl methacrylate, methacrylic acid] (7:3:1 ratio) (e.g., Eudragit® FS30D). In certain preferred embodiments the coating comprises a combination of about 12.9 percent Poly[methacrylic acid, ethyl acrylate] (1:1 ratio) (e.g., Eudragit® L30D-55) and about 51.6 percent Poly[methyl acrylate, methyl methacrylate, methacrylic acid] (7:3:1 ratio) (e.g., Eudragit® FS30D).

In addition to the composition of the coating, the release profile of the dosage form is also influenced by the amount of coating applied to the sealed capsule, referred to herein as the “coating level.” This dimension is expressed herein as the mass of coating per unit area of the capsule, most typically mg/cm² in the context of the dosage forms described herein. In certain embodiments, the coating level is between 5-20 mg/cm². In certain embodiments, the coating level is between 6-16 mg/cm². In certain embodiments, the coating level is between 7-10 mg/cm². In certain embodiments, the coating level is about 6, about 7, about 8, about 9, about 10, about 11, about 12, about 13, about 14, about 15 or about 16 mg/cm². In certain embodiments the coating level is about 6.5, about 7.5, about 8.5, about 9.5, about 10.2, about 10.5, about 10.9, about 11.9, about 12.9, about 13.0 or about 13.7 mg/cm².

The polymer(s) and co-polymer(s) described above are typically provided in the form of aqueous dispersions. A significant component of the enteric coatings described herein therefore is comprised of purified water. In certain embodiments, the water content is between 20-40 percent. In certain preferred embodiments, the water content is about 33 percent.

In certain embodiments, the coating includes other excipients that improve the manufacturability and/or function of the coating. An example of such an excipient that may be included is a plasticizer. Commonly used plasticizers include Propylene glycol, Glycerol, Polyethylene glycols (e.g., PEG-400), Glyceryl triacetate (Triacetin), Triethyl citrate (TEC), Acetyl triethyl citrate, Phthalate esters, Diethyl phthalate, Acetylated, Castor oil and Mineral oil. In certain preferred embodiments, the coating comprises TEC. In certain embodiments, the concentration of TEC is from 1-10 percent. In certain preferred embodiments the TEC content is about 3%.

Another excipient that may be included is a pH modifier, which may be used to adjust the pH at which the polymer(s) or co-polymer(s) dissolves. Examples of such pH modifiers are known by those skilled in the art, and include sodium carbonate, sodium bicarbonate, potassium dihydrogen phosphate and ammonium hydroxide.

Another component that may be added as an excipient in the coating composition and/or added to the outside of the coating is a material used to prevent tackiness during storage. A preferred material for use in this context is talc.

In addition to the effects of the coatings described above, the release profiles of the dosage forms described herein also result from the use of a sealed capsule. The capsules themselves are hard shelled capsules known in the art, commonly made from natural materials such as gelatin, polysaccharide derivatives, such as carrageenans, or celluloses, such as methylcellulose or hydroxypropyl methylcellulose (HPMC). Capsules are comprised of two pieces: a capsule body that is filled with a core composition and a cap that fits over the body. Selection of a particular capsule for use in the dosage forms described herein is within the skill of persons skilled in the art, and will depend in part on the volume of the core composition to be contained therein. In certain preferred embodiments the capsule is comprised of HPMC.

Capsules are typically soluble in gastrointestinal fluids, so in order to avoid release upstream of the region of the intestine at which release is desired, the capsule must be protected from contact with gastrointestinal fluids before reaching that point. Such protection is provided by the use of an appropriate enteric coating as described above, but in order for the enteric coating to provide such protection, it must coat the entirety of the capsule, i.e., with no gaps through which fluid may ingress.

It has been found that the addition of a polymeric seal over the edge of the cap of the capsule, where the cap transitions to the body of the capsule, prior to application of the enteric coating helps avoid release in the proximal small intestine and contributes to improved bioavailability. The amount of surface area of the capsule covered by the seal is not critical, as long as it covers the transition from the cap to the body. Thus, the seal may partially or wholly cover the capsule. For example, in certain embodiments the seal may be a thin band around the capsule, and in other embodiments may be a coat or sub-coat that covers the entirety of the capsule.

In certain preferred embodiments, the polymeric seal is provided through a process known as banding. Banding is typically used on capsule dosage forms filled with liquids to prevent leakage of the liquid interiors, but in the context of the present invention, banding serves to prevent external liquid from reaching the capsule's core composition until the enteric coating has dissolved, as noted above.

Seals for use in dosage form of the present compositions, including bands, may be made from materials having the same dissolution properties as the enteric coating itself, or may have no enteric properties. Seals, including bands, may therefore be made from materials similar to those described above for either the enteric coating or from the same material as the capsule itself. In certain preferred embodiments the band is comprised of the same composition as the enteric coating. In certain preferred embodiments the band is comprised of HPMC. In certain preferred embodiments the band comprises HPMC, ethanol and water. In certain preferred embodiments the band comprises about 17% HPMC, about 58% ethanol and 25% water.

The core compositions of the present invention comprise a therapeutic peptide or protein. The dosage forms described herein may be used for the administration of a variety of therapeutic peptides or proteins, including for example: an incretin therapy with agonist activity at one or more of the glucose-dependent insulinotropic polypeptide (GIP), glucagon-like peptide-1 (GLP-1) and/or glucagon (GCG) receptors; peptide tyrosine-tyrosine (PYY) or an analog thereof; an amylin receptor agonist; or a dual agonist of the amylin and calcitonin receptors. In preferred embodiments the therapeutic peptide or protein is preferably an incretin therapy with agonist activity at the GIP receptor, the GLP-1 receptor and/or the GCG receptor. In certain preferred embodiments, the therapeutic peptide or protein has agonist activity at the GIP receptor and at the GLP-1 receptor. In certain preferred embodiments, the therapeutic peptide or protein has agonist activity at the GLP-1 and GCG receptors. In certain preferred embodiments, the therapeutic peptide has agonist activity at each of the GIP, GLP-1 and GCG receptors. Examples of therapeutic proteins or peptides that may be included in dosage forms of the present invention include semaglutide, the active ingredient in Ozempic® and Rybelsus®, tirzepatide, the active ingredient in Mounjaro®, and other compounds described, for example in WO2020/023386, WO2016/111971, WO2013/164483, WO2019/125938, WO2016/209707, US2020024322, US20210032299, WO2020092191, WO2016034604, WO2015071229, WO2010085700 and WO2022133187.

In certain embodiments, the amount of the therapeutic peptide or protein in the core composition is between 1 and 50 mg. More preferably, the amount of the therapeutic peptide or protein is in the range of 1 to 36 mg. Yet more preferably, the amount of the therapeutic peptide or protein is about 4, 10, 24 or 36 mg.

In certain embodiments, the composition comprises additional excipients, such as a permeation enhancer, a peptidase inhibitor, a lubricant, a filler or bulking agent.

As proteins and peptides are poorly absorbed orally due to low permeability, a permeation enhancer is expected to transiently increase local permeability (Twarog et al. 2019) and result in increased oral bioavailability for therapeutic use. Examples of such permeation enhancers include sodium N-[8-(2-hydroxybenzoyl) amino] caprylate (SNAC), sodium caprylate (C8), sodium caprate or sodium decanoate (C10), or 8-(N-2-hydroxy-5-chlorobenzoyl)-amino-caprylic acid (5-CNAC). Salcaprozate sodium has generally regarded as safe (GRAS) status and is contained in FDA-approved medical food (Eligen®-Vitamin B12, Emisphere, Roseland, NJ, USA). Rybelsus® tablet, which is approved by FDA, European Medicines Agency and Japan Pharmaceuticals and Medical Devices Agency, contains SNAC at 300 mg. C10 has food additive status with no daily limits on consumption, and up to 550 mg per day has been evaluated in an 8-week Phase 2 trial with oral insulin in patients with T2DM with no safety concerns reported (Halberg et al. 2019).

In certain preferred embodiments, the permeation enhancer is C10. In certain preferred embodiments the core composition comprises between 1-500 mg C10. In certain preferred embodiments the core composition comprises between 200-300 mg C10. In certain preferred embodiments the core composition comprises about 280 mg C10.

The core composition of dosage forms of the present invention optionally further comprise a capsule filler to improve flow, compactability, tackiness, and density of the blend. A preferred example of such a material is microcrystalline cellulose (MCC). In certain preferred embodiments, dosage forms of the present invention comprise MCC. In certain preferred embodiments, core compositions of the present invention comprise up to 100 mg MCC. In certain preferred embodiments, core compositions of the present invention comprise about 66 mg MCC.

The composition may include additional functional excipients, such as peptidase inhibitors, as well as common tableting/encapsulation excipients such as a lubricant (e.g., magnesium stearate, sodium stearyl fumarate (SFF)), disintegrant (sodium starch glycolate, crospovidone, croscarmellose sodium, starch, etc.) and/or glidant (colloidal silica, starch, silicone oil, talc, etc.).

An advantage of dosage forms of the present invention as compared to oral dosage forms known in the art is improved bioavailability of the therapeutic peptide or protein. Bioavailability may be measured by procedures known in the art, including for example those described in the examples below. Oral bioavailability may in some contexts be compared to absolute bioavailability, which refers to area under the curve (AUC) of the pharmacokinetic profile of the therapeutic peptide or protein following intravenous (IV) administration. In some contexts, however, it may be preferred to identify a relative bioavailability of oral AUC as compared, for example, to AUC following subcutaneous (SC) administration. The bioavailability of the therapeutic peptide or protein is thus typically measured by comparing the AUC of the pharmacokinetic profile of the therapeutic peptide or protein after oral administration to the AUC after parenteral administration of the therapeutic peptide or protein. Bioavailability in this context is commonly expressed as the fraction—or percent F—of the drug's AUC achieved following oral administration.

Dosage forms of the present invention result in bioavailability >1% F following oral administration relative to SC administration. Preferably, dose forms of the present invention result in a bioavailability of >3% F, and more preferably >4% F following oral administration relative to SC administration. In certain preferred embodiments, dosage forms of the present invention result in bioavailability of up to 10% F following oral administration relative to SC administration.

Another advantage of dosage forms of embodiments described herein is that they may achieve acceptable bioavailability regardless of whether they are administered while the patient is in a fed state or a fasted state. When used herein, the term “fed state,” also known as the absorptive state, refers to the condition of a person's body after they have eaten food and the body is digesting the food and absorbing the nutrients. This condition begins when food is eaten, and may remain for up to about 4 hours depending on the types and amounts of nutrients ingested. The term “fasted state,” by contrast refers to the condition of a person's body when it is not digesting food and absorbing nutrients. As the duration of the fed state varies, drugs requiring administration during a fasted state are commonly required to be taken in the morning, before breaking the nighttime fast. For example, the oral presentation of semaglutide RYBELSUS® is required to be taken at least 30 minutes before the first food, beverage, or other oral medications of the day with no more than 4 ounces of plain water only. Waiting less than 30 minutes, or taking with food, beverages (other than plain water) or other oral medications will lessen the effect of RYBELSUS®. Preferred dosage forms of the present invention, by contrast, may be taken any time before, during or after eating, drinking or taking other oral medications with little or no lessening of their effects. In certain embodiments, negative food effects on bioavailability are not observed when dosage forms of the present invention are co-administered with food.

Dosage forms of the present invention may be used in the treatment of a range of diseases or disorders. When the therapeutic peptide or protein is an incretin therapy, dosage forms of the present invention may be used to treat one or more diseases selected from the group consisting of type 2 diabetes mellitus (T2DM), obesity, cardiovascular disease (CVD), non-alcoholic steatohepatitis (NASH), fatty liver disease (FLD), dyslipidemia, metabolic syndrome, cognitive decline, Parkinson's syndrome and Alzheimer's disease. In certain preferred embodiments the disease or condition is T2DM. In certain preferred embodiments the disease or condition is obesity. In certain preferred embodiments dosage forms of the present invention may be used to provide non-therapeutic weight loss.

When used in the treatment of diseases or conditions such as those described above, dosage forms of the present invention may be administered once daily, twice daily, alternate days, every third day, every fourth day, every fifth day, every sixth day or once weekly. Preferably, the dosage forms are administered once daily.

When used herein, the terms “treatment,” “treat,” “treating” nd the like, are meant to include slowing or attenuating the progression of a disease or disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

When used herein in the context of quantitation of the amount of therapeutic protein or peptide released from a dosage form in a fluid of a given pH, the term “majority refers to release of greater than 50% of the therapeutic protein or peptide included in the dosage form when it is prepared. The term “none” means that the dosage form releases either 0% or a de minimis amount of the therapeutic protein or peptide included in the dosage form when it is prepared. As used herein, the term “about” is intended to refer to an acceptable degree of error for the amount or quantity indicated given the nature or precision of the measurements. For example, the degree of error can be indicated by the number of significant FIGURES provided for the measurement, as is understood in the art, and includes but is not limited to a variation of +/−1 in the most precise significant FIGURE reported for the amount or quantity. Typical exemplary degrees of error are within 20 percent (%), preferably within 10%, and more preferably within 5% of a given value or range of values. Numerical quantities given herein are approximate unless stated otherwise, meaning that the term “about” can be inferred when not expressly stated.

Examples of the aforementioned dosage forms, their method of preparation, and uses are included in the studies described below.

### COMPOUNDS USED IN EXAMPLES

### Compound 1

### Y-Aib-EGT-αMeF(2F)-TSD-4Pal-SI-αMeL-LD-Orn-K((2-[2-(2-Amino-ethoxy)-ethoxy]-acetyl)₂-(γ-Glu)-CO—(CH₂)₁₆—CO₂H)AQ-Aib-EFI-(D-Glu)-αMeY-LIEGGPSSGAPPPS-NH₂ (SEQ ID NO:1)

The structure of SEQ ID NO:1 is depicted below using the standard single letter amino acid codes with the exception of residues Aib2, αMeF(2F)6, 4Pal10, αMeL13, Om16, K17, Aib20, D-Glu24 αMeY25, and Ser39, where the structures of these amino acid residues have been expanded:

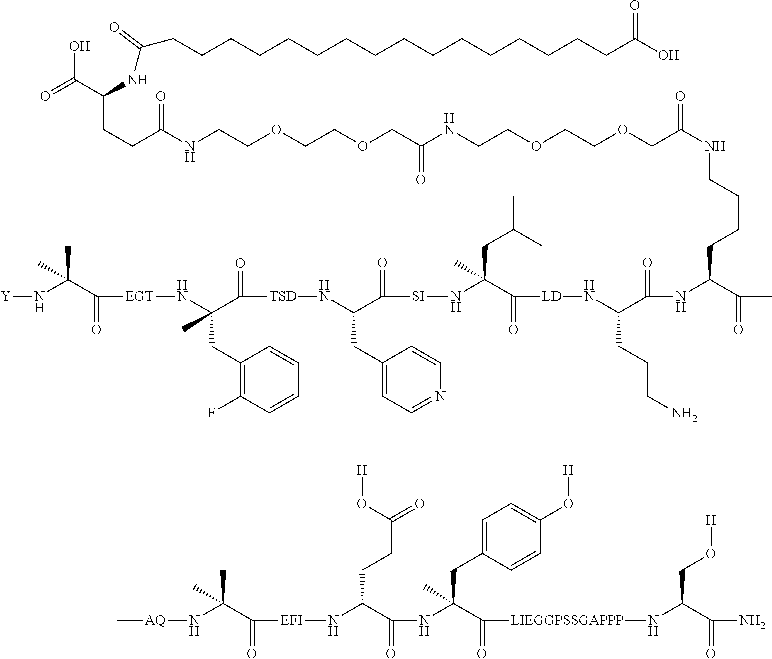

The compound according to SEQ ID NO:1 is prepared substantially as described in US2020024322.

### Compound 2

### Y-Aib-EGT-αMeF(2F)-TSDYSI-αMeL-LD-Orn-K((2-[2-(2-Amino-ethoxy)-ethoxy]-acetyl)₂-(γ-Glu)-CO—(CH₂)₁₆—CO₂H)AQ-Aib-EFI-(D-Glu)-YLIEGGPSSGAPPPS-NH₂ (SEQ ID NO:2)

The structure of SEQ ID NO:2 is depicted below using the standard single letter amino acid codes with the exception of residues Aib2, αMeF(2F)6, αMeL13, Orn16, K17, Aib20 D-Glu24, and Ser39 where the structures of these amino acid residues have been expanded:

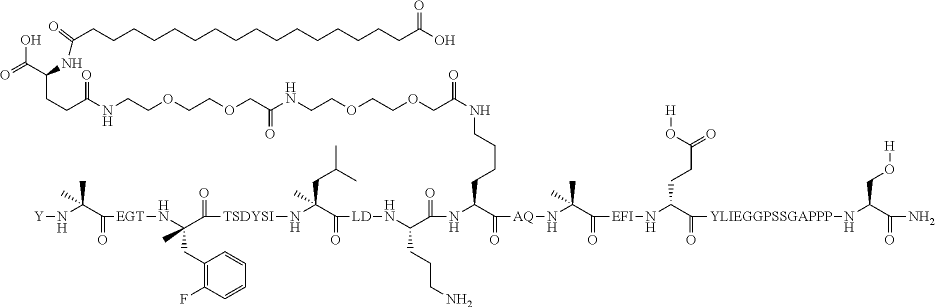

The compound according to SEQ ID NO:2 is prepared substantially as described in US2020024322.

### Compound 3

### YAibEGTFTSDYSIAibLDKIAQK(([2-(2-Amino-ethoxy)-ethoxy]-acetyl)₂-(γGlu)₂-CO—(CH₂)₁₈—CO₂H)A-1-Nal-VQWLIAGGPSSGAPPPS-NH₂ (SEQ ID NO:3)

The structure of SEQ ID NO:3 is depicted below using the standard single letter amino acid codes with the exception of residues Aib2, Aib13, K(20 and 1-Nal22 where the structures of these amino acid residues have been expanded.

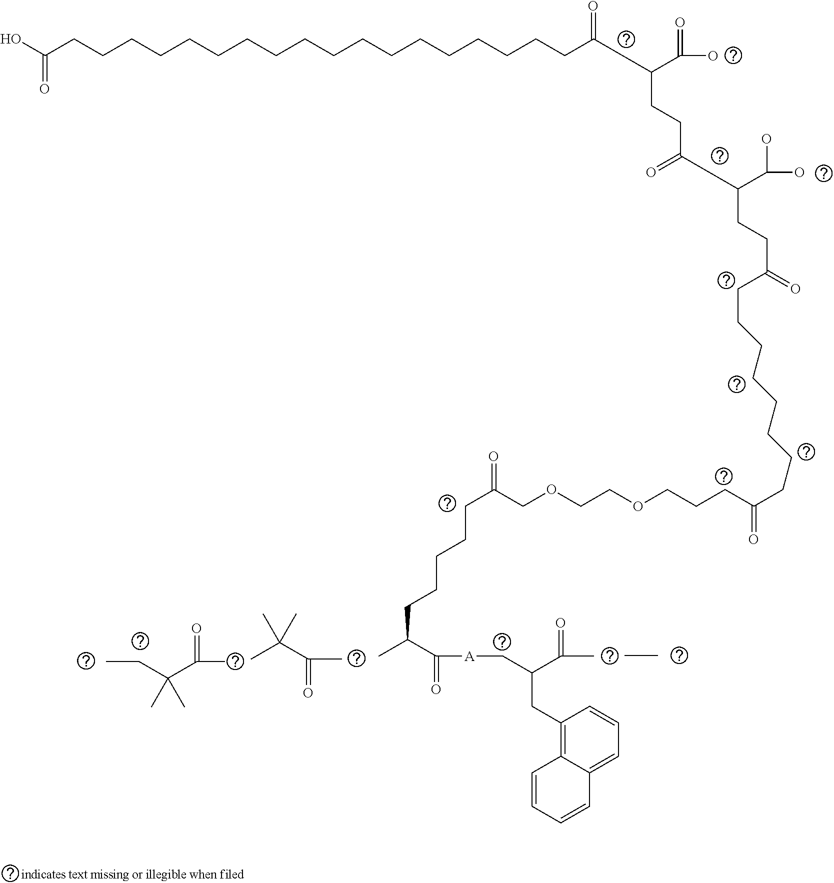

The peptide according to SEQ ID NO: 3 may be prepared substantially as described in US2016/0199438.

### Tirzepatide

### YAibEGTFTSDYSTAibLDKTAQK((12-(2-Amino-ethoxy)-ethoxy]-acetyl)₂-(γGlu)1-CO—(CH₂)₁₈—CO₂H)AFVQWLIAGGPSSGAPPPS-NH₂ (SEQ ID NO: 4)

The structure of SEQ ID NO: 4 is depicted below using the standard single letter amino acid code with exception of residues Aib2, Aib13 and K2O where the structures of these amino acid residues have been expanded.

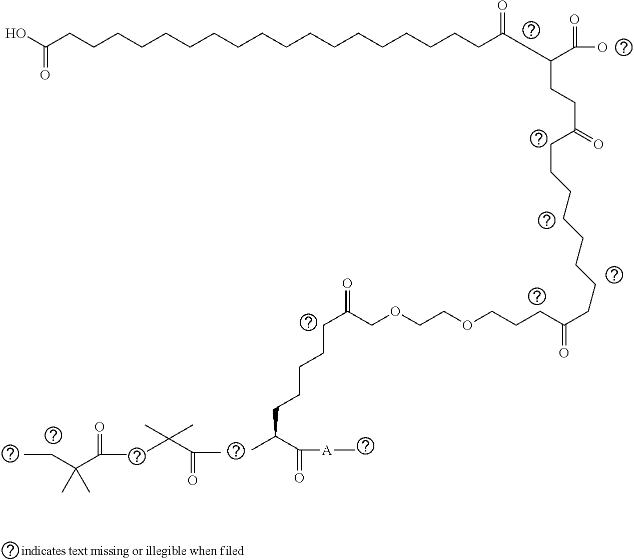

The peptide according to SEQ ID NO: 4 may be prepared substantially as described in US2016/0199438.

### Examples 1-6

Capsules prepared by blending the compound and C10 amounts set forth in Tables 3A and 3B below in a suitable blender (at small-scale, all the components are weighed and transferred into a jar and blended for 5 mins at 25 G in a resonant acoustic mixer), weighing the target blend, compressing the blend into a slug using an appropriate capsule slug mold, and placing it to fit inside an enteric capsule body. Examples 1-2 and 5-6 are prepared with Compound 1, Example 3 is prepared with Compound 2 and Example 4 is prepared with Compound 3. In some of the Tables herein, these compounds are referred to as “LY.”

The filled capsules are then coated using a pan coater with coating system I or II described in Table 2 to different levels of coating expressed in mg/cm². The composition and coating levels are shown in Tables 3A and 3B. After coating, each capsule is manually sealed using 10 uL coating solution around cap and body transition area, allowed to dry and then stored refrigerated. The blending conditions, encapsulation/coating/sealing processes are further modified upon scale-up to achieve optimum manufacturability and required capsule attributes depending upon the scale and manufacturing equipment utilized.

Dissolution testing of capsules is performed with a USP Apparatus II using paddles and 1 L vessels. The dissolution test has two or three consecutive stages of media challenge, where the capsule is placed in an appropriately sized sinker and transferred to pre-warmed media (37 °C) for each stage. Stage 1 is conducted in 750 mL of pH 4.5 acetate buffer with a paddle speed of 100 rpm for 4 hours. Stage 2 is conducted in 750 mL of 50 mM pH 6.0 phosphate buffer with a paddle speed of 75 rpm for 2 hours. Stage 3 is conducted in 750 mL of 50 mM pH 6.8 phosphate buffer with a paddle speed of 75 rpm for at least 2 hours. Coating system I is tested with Stages 1 and 2. Coating system II is tested with Stages 1, 2, and 3. Release of the peptide and C10 is determined by UV absorption on a suitable HPLC system.

Table 4 shows dissolution results for capsules coated with coating system I described in Compositions 1, and 4. Table 5 shows dissolution results for capsules coated with coating system II described in Compositions 1, 5, and 6.

### Example 7

Capsules are prepared by blending the compound and C10 amounts set forth in Tables 3A and 3B below in a suitable blender, weighing the target blend, compressing the blend using an appropriate capsule slug mold, and placing it into a slug to fit inside an HPMC capsule body. The filled capsules are banded with an HPMC banding using lab-scale banding equipment. The composition of the banding solution is shown in Table 7. After banding, capsules are coated using a pan coater with coating system I or II described in Table 2 to different levels of coating expressed in mg/cm². The blending conditions, encapsulation/banding/coating processes are further modified upon scale-up to achieve optimum manufacturability and required capsule attributes depending upon the scale and manufacturing equipment utilized.

Dissolution testing is performed with a USP Apparatus II using paddles and 1L vessels. The dissolution test has two or three consecutive stages of media challenge, where the capsule is placed in an appropriately sized sinker and transferred to pre-warmed media (37 °C) for each stage. Stage 1 is conducted in 750 mL of pH 4.5 acetate buffer with a paddle speed of 100 rpm for 4 hours. Stage 2 is conducted in 750 mL of 50 mM pH 6.0 phosphate buffer with a paddle speed of 75 rpm for 2 hours. Stage 3 is conducted in 750 mL of 50 mM pH 6.8 phosphate buffer with a paddle speed of 75 rpm for at least 2 hours. Coating system I is tested with Stages 1 and 2. Coating system II is tested with Stages 1, 2, and 3. Release of the peptide and C10 is determined by UV absorption on a suitable HPLC system.

Table 7 shows dissolution results for capsules coated with coating system I from Example 7 and Table 9 shows dissolution results for capsules coated with coating system II from Example 7.

TABLE 2
Dosage form of Coating Suspensions.

| | Coating | Coating |
|---|---|---|
| | System I | System II |
| Component | % w/w | % w/w |
| L30D-55 | 64.5 | 12.9 |
| FS30D | — | 51.6 |
| TEC | 2.9 | 2.9 |
| Purified water | 32.6 | 32.6 |

TABLE 3A
Composition for Capsule Formulation Examples 1-4.

| | Example 1 | Example 2 | Example 3 | Example 4 |
|---|---|---|---|---|
| Compound (mg) | 20 | 20 | 20 | 20 |
| C10 (mg) | 280 | 280 | 280 | 280 |
| Total | 302.55 | 302.55 | 302.81 | 303.41 |
| Capsule | | | | |
| Weight | | | | |
| Target (mg) | | | | |

| Coating systems | I | II | I | II | I | II | I | II |
|---|---|---|---|---|---|---|---|---|
| Coating | 12.9 | 10.9 | N/A | 12.4 | N/A | 13 | 13 | 11.9 |
| level(s) | | | | | | | | |
| (mg/cm²) | | | | | | | | |

TABLE 3B
Composition for Capsule Formulation Examples 5-7.

| | Example 5 | Example 6 | Example 7 |
|---|---|---|---|
| Compound (mg) | 20 | 20 | 4 |
| C10 (mg) | 280 | 280 | 280 |
| Total | 302.55 | 302.55 | 284 |
| Capsule | | | |
| Weight | | | |
| Target (mg) | | | |

| Coating systems | I | II | I | II | I | II |
|---|---|---|---|---|---|---|
| Coating | N/A | 13 | N/A | 13.7 | 7-16 | 7-10 |
| level(s) | | | | | | |
| (mg/cm²) | | | | | | |

TABLE 4
Results of Dissolution Testing for Capsules
Coated with Coating System I.

| Example 1 | | Example 4 | |
|---|---|---|---|
| 12.9 mg/cm² Coating | | 13.0 mg/cm² Coating | |
| system I | | system I | |
| Time | % Release | Time | % Release |

| (min) | LY | C10 | (min) | LY | C10 |
|---|---|---|---|---|---|
| 0 | 0.0 | 0.0 | 0 | 0.0 | 0.0 |
| 240 | 0.0 | 0.0 | 240 | 0.0 | 0.0 |
| 255 | 0.2 | 0.0 | 250 | 0.0 | 0.0 |
| 270 | 0.4 | 0.0 | 260 | 2.3 | 1.7 |
| 285 | 72.7 | 72.3 | 270 | 62.8 | 62.2 |
| 300 | 100.2 | 97.6 | 280 | 96.4 | 94.8 |
| 330 | 101.8 | 97.9 | 290 | 100.7 | 98.0 |
| 360 | 100.9 | 98.5 | | | |

As seen in Table 4, coating system I results in no release after 240 min in pH 4.5 and complete release of LY and C10 within 120 additional minutes in pH 6.0. The aforementioned data are consistent with supporting no release in the gastric compartment and release in the middle small intestine.

TABLE 5
Results of Dissolution Testing for Capsules
Coated with Coating System II.

| Example 1 | | | Example 5 | | | Example 6 | | |
|---|---|---|---|---|---|---|---|---|
| 10.9 mg/cm² | | | 13.0 mg/cm² | | | 13.7 mg/cm² | | |
| Coating | | | Coating | | | Coating | | |
| system II | | | system II | | | system II | | |
| Time | % Release | | Time | % Release | | Time | % Release | |
| (min) | LY | C10 | (min) | LY | C10 | (min) | LY | C10 |
| 0 | 0.0 | 0.0 | 0 | 0.0 | 0.0 | 0 | 0.0 | 0.0 |
| 270 | 0.0 | 0.0 | 240 | 0.0 | 0.0 | 240 | 0.0 | 0.0 |
| 300 | 0.0 | 0.0 | 360 | 0.0 | 0.0 | 360 | 0.0 | 0.0 |
| 330 | 0.0 | 0.0 | 390 | 0.0 | 0.0 | 645 | 0.0 | 0.0 |
| 360 | 0.0 | 0.0 | 420 | 0.0 | 0.0 | | | |
| 375 | 0.0 | 0.0 | 450 | 0.0 | 0.0 | | | |
| 390 | 0.0 | 0.0 | 465 | 0.0 | 0.0 | | | |
| 405 | 0.0 | 0.0 | 480 | 0.0 | 0.1 | | | |
| 420 | 0.0 | 0.0 | 495 | 0.0 | 0.0 | | | |
| 435 | 0.0 | 0.0 | 505 | 4.9 | 1.8 | | | |
| 450 | 0.0 | 0.0 | 515 | 6.2 | 5.3 | | | |
| 465 | 0.0 | 0.0 | 525 | 6.4 | 4.4 | | | |
| 480 | 0.0 | 0.0 | 535 | 7.3 | 2.8 | | | |
| 510 | 1.6 | 0.0 | 545 | 10.4 | 7.0 | | | |
| 520 | 1.8 | 0.0 | 555 | 66.9 | 61.8 | | | |
| 540 | 32.6 | 30.9 | 565 | 100.4 | 92.7 | | | |
| 560 | 66.3 | 65.1 | 575 | 105.1 | 95.4 | | | |
| 570 | 82.5 | 79.7 | 585 | 106.7 | 97.0 | | | |
| 585 | 91.8 | 89.7 | | | | | | |

As seen in Table 5, Examples 1 and 5, coating system II results in no release after 240 min in pH 4.5, minimal or no release after 120 additional minutes in pH 6.0, and complete release of LY and C10 within 225 additional minutes in pH 6.8. These data are consistent with supporting no release in the gastric compartment and full release after reaching the lower small intestine. As seen in Table 5, 13.7 mg/cm² of coating system II results in no release after 645 minutes which may correspond to minimal or no release in vivo.

TABLE 6
Composition of Banding Solution for Example 7.

| Component | % w/w |
|---|---|
| HPMC | 14 |
| Ethanol | 60 |
| Water | 26 |

TABLE 7
Results of Dissolution Testing for Capsules Described
in Example 7 coated with Coating System I.

| Example 7, | | | Example 7, | | | Example 7, | | |
|---|---|---|---|---|---|---|---|---|
| 7 mg/cm² | | | 12 mg/cm² | | | 16 mg/cm² | | |
| Coating | | | Coating | | | Coating | | |
| system I | | | system I | | | system I | | |
| Time | % Release | | Time | % Release | | Time | % Release | |
| (min) | LY | C10 | (min) | LY | C10 | (min) | LY | C10 |
| 0 | 0.0 | 0.0 | 0 | 0.0 | 0.0 | 0 | 0.0 | 0.0 |
| 240 | 0.0 | 0.0 | 240 | 0.0 | 0.1 | 240 | 0.0 | 0.0 |
| 245 | 0.0 | 0.1 | 255 | 0.0 | 0.0 | 255 | 0.0 | 0.0 |
| 250 | 8.3 | 9.4 | 260 | 0.0 | 0.0 | 270 | 0.0 | 0.0 |
| 255 | 35.0 | 35.0 | 265 | 0.8 | 0.4 | 280 | 0.0 | 0.0 |
| 260 | 67.5 | 62.8 | 270 | 8.3 | 9.4 | 290 | 7.5 | 8.5 |
| 270 | 104.2 | 93.9 | 275 | 35.0 | 32.8 | 300 | 45.0 | 44.6 |
| | | | 280 | 70.0 | 62.8 | 310 | 90.8 | 91.4 |
| | | | 285 | 99.2 | 87.0 | 320 | 105.0 | 103.1 |

As seen in Table 7, coating system I results in no or minimal release after 240 min in pH 4.5 and complete release of LY and C10 within 120 additional minutes in pH 6.0 with delay in release as the coating level increased from 7 mg/cm² to 16 mg/cm². These data are consistent with supporting no release in the gastric compartment and release in the middle small intestine.

TABLE 8
Results of Dissolution Testing for Capsules Described
in Example 7 coated with Coating System II.

| Example 7 | | Example 7, | |
|---|---|---|---|
| 7 mg/cm² Coating | | 10 mg/cm² Coating | |
| system II | | system II | |
| Time | % Release | Time | % Release |

| (min) | LY | C10 | (min) | LY | C10 |
|---|---|---|---|---|---|
| 0 | 0.0 | 0.0 | 0 | 0 | 0 |
| 240 | 0.0 | 0.0 | 240 | 0.0 | 0.0 |
| 300 | 0.0 | 0.0 | 360 | 0.0 | 0.0 |
| 330 | 0.0 | 0.0 | 390 | 0.0 | 0.0 |
| 345 | 0.0 | 0.0 | 450 | 26.3 | 25.1 |
| 360 | 0.0 | 0.0 | 470 | 32.5 | 31.8 |
| 365 | 9.2 | 10.0 | 495 | 74.4 | 71.4 |
| 370 | 23.3 | 23.4 | 515 | 80.6 | 76.7 |
| 375 | 38.3 | 38.1 | | | |
| 380 | 49.2 | 47.3 | | | |
| 390 | 60.0 | 58.2 | | | |

As seen in Table 8, coating system II results in no release after 240 min in pH 4.5, minimal or no release after 120 additional minutes in pH 6.0, and complete release of LY and C10 within 155 additional minutes in pH 6.8 with a delay in release as the coating level increases from 7 mg/cm² to 10 mg/cm². These data are consistent with supporting no release in the gastric compartment and full release after reaching the lower small intestine.

### Example 8

Banded and coated capsules comprising C10 and tirzepatide are prepared following the general description described above for Example 7. Details of the dosage forms are set forth in Table 9 below.

TABLE 9
TZP formulation details: banded, coated capsule.

| Component | mg/capsule |
|---|---|
| tirzepatide (mg) | 20 |
| C10 (mg) | 280.00 |
| Total Capsule Weight Target (mg) | 304.51 |
| Coating systems | II |
| Coating level(s) (mg/cm²) | 10.2 |

Dissolution testing performed following the general description described above for Examples 1-7. Table 10 shows dissolution results for the dosage forms of Example 8.

TABLE 10
Dissolution results for Example 8.
Example 8 10.2 mg/cm² Coating system II

| | |
|---|---|
| Time | % Release |

| (min) | LY | C10 |
|---|---|---|
| 0 | 0.0 | 0.0 |
| 240 | 0.0 | 0.1 |
| 330 | 0.0 | 0.0 |
| 360 | 0.0 | 0.0 |
| 375 | 28.8 | 33.6 |
| 385 | 78.5 | 78.4 |
| 395 | 94.9 | 93.8 |
| 405 | 98.3 | 95.2 |
| 415 | 99.6 | 95.9 |
| 425 | 100.8 | 96.7 |
| 435 | 100.6 | 97.1 |
| 450 | 101.8 | 97.7 |
| 470 | 102.2 | 98.2 |

As seen in Table 10, coating system II results in no release after 240 min in pH 4.5, minimal or no release after 120 additional minutes in pH 6.0, and complete release of TZP and C10 within 110 additional minutes in pH 6.8. These data are consistent with supporting no release in the gastric compartment and full release after reaching the lower small intestine.

### Reference Dosage Form

Capsules are coated with using a fluid bed coater with coating system described in Table 11. After coating, each capsule is manually opened to accept powder fill. The capsules for use in in vivo testing are prepared by individually weighing and transferring the Compound 1 and C10 amounts set forth in Table 12 below into the enteric coated capsules. The capsules for use in dissolution testing are prepared by blending the Compound 1 and C10 amounts set forth in Table 12 below in a suitable blender, then filling the target blend amount into the enteric coated capsules. Capsules for dissolution tests are stressed at 40 degree c/10% RH for 1 month before testing.

Dissolution testing A of capsules is performed with a USP Apparatus II using paddles and 1 L vessels. The dissolution test has two consecutive stages of media challenge, where the capsule is placed in an appropriately sized sinker and transferred to pre-warmed media (37 °C) for each stage. Stage 1 is conducted in 750 mL of pH 2.0 HCl with 0.1% Tween-80 at a paddle speed of 100 rpm for 2 hours. Stage 2 is conducted in 1000 mL of 50 mM pH 6.8 phosphate buffer with 0.1% Tween 80 at a paddle speed of 75 rpm for 30 mins. Release of the peptide and C10 is determined by UV absorption on a suitable HPLC system.

Dissolution testing B of capsules is performed with a USP Apparatus II using paddles and 1 L vessels. The capsule is placed in an appropriately sized sinker and transferred to pre-warmed media (37 °C). Dissolution testing B is conducted in 750 mL of pH 4.5 Acetate buffer with 0.10% Tween-80 at a paddle speed of 100 rpm for 2 hours. Release of the peptide and C10 is determined by UV absorption on a suitable HPLC system. Tables 13 and 14 show dissolution results for capsules described in this study.

TABLE 11
Composition of Coating Suspensions.

| | Coating |
|---|---|
| Component | System I |
| Acryl-EZE | 18.18 |
| FS30D | — |
| TEC | 1.80 |
| Anti-foaming agent | 0.02 |
| Purified water | 80.00 |

TABLE 12
Composition for Capsule Formulation in Reference Dosage Form.

Amount per Capsule

| | animal study | dissolution test |
|---|---|---|
| Compound (mg) | 10.00 | 4.00 |
| C10 (mg) | 250.00 | 250.00 |
| Microcrystalline Cellulose | — | 50 |
| Magnesium Stearate | — | 5.5 |
| Total Capsule Weight Target | 260.00 | 309.5 |
| (mg) | | |
| Coating level(s) | 4.99 | 4.99 |
| (mg/cm²) | | |

TABLE 13
Results of Dissolution Testing A for Capsules.

| | |
|---|---|
| Time | % Release |

| (min) | LY | C10 |
|---|---|---|
| 0 | 0.0 | 0.0 |
| 30 | 0.0 | 0.0 |
| 60 | 0.0 | 0.0 |
| 90 | 0.0 | 0.0 |
| 120 | 0.0 | 0.0 |
| 125 | 5.8 | 8.8 |
| 129 | 86.7 | 97.6 |
| 135 | 91.3 | 101.6 |
| 150 | 91.2 | 101.8 |

TABLE 14
Results of Dissolution Testing B for Capsules.

| | |
|---|---|
| Time | % Release |

| (min) | LY | C10 |
|---|---|---|
| 0 | 0.0 | 0.0 |
| 30 | 0.0 | 0.0 |
| 60 | 0.0 | 0.0 |
| 90 | 0.0 | 0.0 |
| 120 | 1.8 | 1.8 |

As seen in Table 13, the reference dosage form capsules result in no release after 120 min in pH 2.0 and complete release of Compound 1 and C10 within 30 additional minutes in pH 6.8. Additionally, capsules show release starts at 2 hrs in pH 4.5 in table 14. The aforementioned data support no release in the gastric compartment and release in the upper small intestine.

### Pharmacokinetics of Compound 1 in Beagle Dogs:

### Subcutaneous (SC) Administration

Compound 1 and the internal standard are extracted from dog plasma (50 μL) by protein precipitation using isopropyl alcohol and methanol (50:50 v/v). The samples are then centrifuged (4000 rpm for 10 minutes) and the supernatant is transferred to a Siricco Protein Precipitation Plate. After centrifugation (4000 rpm for 20 minutes), the samples are loaded on a Sep-Pak tC18 SPE microelution plate that is conditioned with 2% formic acid in water. The compounds are then washed with 2% formic acid in water and eluted using 2% formic acid in acetonitrile into a plate containing 1× Invitrosol and 1% formic acid in water prior to injecting an aliquot (20 μL) on to Advantage Armor C18, 3 μm, 30×0.5 mm for LC/MS analysis.

Blood samples are collected over 336 hours. Plasma is harvested from blood samples by centrifugation and stored frozen (—60 to −80 °C) until analysis. Plasma concentrations of Compound 1 are detected according to the bioanalytical method described above.

PK parameters after a single SC dose of Compound 1 are presented in Table 15.

TABLE 15
Individual and Mean Pharmacokinetic Parameters
Following a Single SC Dose of Compound 1 (0.020
mg/kg or 4.06 nmol/kg) to Beagle Dogs.

| | T_{1/2} | Tₘₐₓ | Cₘₐₓ | AUC_{0-inf} | CL/F |
|---|---|---|---|---|---|
| N = 4 | (hr) | (hr) | (nmol/L) | (hr*nmol/L) | (mL/hr/kg) |
| Mean | 68 | 12 | 33.0 | 3589 | 1.14 |
| SD | 3 | 0 | 3.9 | 298 | 0.09 |

Abbreviations: AUC_{0-inf} = area under the curve from time 0 hours to infinity; CL/F = apparent clearance; Cₘₐₓ = maximal concentration; Tₘₐₓ = time to maximal concentration; T_{1/2} = half-life.

### Oral Administration in Dosage Forms Targeting Proximal, Middle and Distal Intestine Administration

Compound 1 and the internal standard are extracted from dog plasma (50 μL) by antibody capture using biotinylated antibodies IBA395 and IBA5C9 (1:1, 2 μg/well). Samples are mixed on a plate shaker for 1 hour before adding 20 μL of high-capacity magnetic beads. Samples are then mixed for 30 minutes before washing twice with phosphate buffered saline and eluting with 100 μL of 1% formic acid in water and acetonitrile (70/30% v/v). An aliquot (20 μL) is injected on to 2× Sprite AC1842 C18, 5 μm, 40×2.1 mm for LC/MS analysis.

PK parameters of Compound 1 are determined after a single 20 mg oral dose of Compound 1 in different formulations to male beagle dogs following overnight fasting. Blood samples are collected up to 168 hours post-dose. Plasma is harvested from blood samples by centrifugation and stored frozen (−60 to −80 °C) until analysis. Plasma concentrations of Compound 1 are detected through 168 hours post-dose.

PK parameters after a single oral dose of Compound 1 to beagle dogs are presented in Table 16.

TABLE 16
Individual and Mean Pharmacokinetic Parameters Following a Single Oral Dose
of Compound 1 (20 mg) with Sodium Caprate (C10, 280 mg) to Beagle Dogs.

| | | | | | | | F (AUC, |
|---|---|---|---|---|---|---|---|
| | | | | | | | PO/AUC, |
| | | T_{1/2} | Tₘₐₓ | Cₘₐₓ | AUC_{0-inf} | CL/F | SC) |
| Formulation | | (hr) | (hr) | (nmol/L) | (hr*nmol/L) | (mL/hr/kg) | (%) |
| Reference | Mean | 64 | 4 | 15 | 980 | 673 | 0.29 |
| (n = 7)^{a} | SD | 11 | 4 | 10 | 577 | 615 | 0.17 |
| Example 1, | Mean | 77 | 2.3 | 227 | 13112 | 38.5 | 4.2 |
| System I | SD | 22 | 0.8 | 115 | 8715 | 24.5 | 2.8 |
| (n = 7)^{b} | | | | | | | |
| Example 1, | Mean | 76 | 3.0 | 401 | 24183 | 16.5 | 7.8 |
| System II | SD | 13 | 1.1 | 192 | 11377 | 4.9 | 3.7 |
| (n = 8)^{c} | | | | | | | |

^{a}Dose = 386.4 nmol/kg;
^{b}Dose = 350.4 nmol/kg;
^{c}Dose = 352.5 nmol/kg;
Abbreviations: AUC_{0-inf} = area under the curve from time 0 hours to infinity; CL/F = apparent clearance; Cₘₐₓ = maximum concentration; Tₘₐₓ = time to maximal concentration; T_{1/2} = half-life; F (AUC, PO/AUC, SC) = relative bioavailability.

As seen in Table 16, oral administration of reference dosage form capsules resulted in mean bioavailability of 0.29% of the bioavailability observed when administered Compound 1 was administered subcutaneously, oral administration of Example 1, System I resulted in mean bioavailability of 4.2% of the bioavailability observed when Compound 1 was administered subcutaneously, and oral administration of Example 1, System II resulted in mean bioavailability of 7.8% of the bioavailability observed when Compound 1 was administered subcutaneously.

Oral Administration with Food in Dosage Form Targeting Distal Intestine Release

The effect of food is evaluated following a single 20 mg dose of Compound 1 administered orally to male beagle dogs. The dosage forms are prepared as described above for Example 2. The animals are fasted overnight prior to dosing and are provided food either right after dosing (1-minute post-dose) or 30 minutes post-dose. Blood samples are collected up to 168 hours post-dose. Plasma is harvested from blood samples by centrifugation and stored frozen (−50 to −100 °C) until analysis. Plasma concentrations of Compound 1 are detected through 168 hours post-dose. PK parameters after a single oral dose of Compound 1 administered in the presence of food to beagle dogs are presented in Table 17.

TABLE 17
Individual and Mean Pharmacokinetic Parameters Following a Single Oral Dose of Compound
1 (20 mg) with Sodium Caprate (C10, 280 mg) in the presence of food to Beagle Dogs.

| | | | | | | | | F (AUC, |
|---|---|---|---|---|---|---|---|---|
| | | | | | | | | PO/AUC, |
| | Dose | Animal | T_{1/2} | Tₘₐₓ | Cₘₐₓ | AUC_{0-inf} | CL/F | SC) |
| Food timing | (nmol/kg) | ID | (hr) | (hr) | (nmol/L) | (hr*nmol/L) | (mL/hr/kg) | (%) |
| 1-minute post- | 375.7 | Mean | 46 | 11 | 511 | 28168 | 35.2 | 8.5 |
| dose (n = 8) | | SD | 15 | 6 | 385 | 27628 | 45.1 | 8.3 |
| 0.5 hours post- | 377.1 | Mean | 55 | 9 | 791 | 38910 | 12.7 | 11.7 |
| dose (n = 6) | | SD | 16 | 2 | 549 | 22905 | 6.7 | 6.9 |

Abbreviations: AUC_{0-inf} = area under the curve from time 0 hours to infinity; CL/F = apparent clearance; Cₘₐₓ = maximum concentration; Tₘₐₓ = time to maximal concentration; T_{1/2} = half-life; F (AUC, PO/AUC, SC) = relative bioavailability.

As seen in Table 17, oral administration of Example 2 when dogs were fed 1-minute and 0.5 hours resulted in mean bioavailability of 8.5% and 11.7%, respectively, of the bioavailability observed when Compound 1 was administered subcutaneously.

### Pharmacokinetics of Compound 2 in Beagle Dogs

### Bioanalytical Summary

Compound 2 and the internal standard are extracted from dog plasma (50 μL) by antibody capture using biotinylated antibodies IBA395 and IBA5C9 (1:1, 2 μg/well). Samples are mixed on a plate shaker for 1 hour before adding 20 μL of high-capacity magnetic beads. Samples are then mixed for 30 minutes before washing twice with phosphate buffered saline and eluting with 100 μL of 1% formic acid in water and acetonitrile (70/30% v/v). An aliquot (20 μL) is injected on to 2× Sprite AC1842 C18, 5 μm, 40×2.1 mm for LC/MS analysis.

### Intravenous Administration

The plasma pharmacokinetics (PK) of Compound 2 are evaluated in male beagle dogs following a single intravenous (IV) dose (0.02 mg/kg or 4.06 nmol/kg). Blood samples are collected over 336 hours. Plasma is harvested from blood samples by centrifugation and stored frozen (−50 to −100 °C) until analysis. Plasma concentrations of Compound 2 are detected according to the bioanalytical method described above. PK parameters after a single IV dose of Compound 2 are presented in Table 18.

TABLE 18
Individual and Mean Pharmacokinetic Parameters
Following a Single IV Dose of Compound 2 (0.020
mg/kg or 4.06 nmol/kg) to Beagle Dogs.

| | T_{1/2} | AUC_{0-inf} | CL |
|---|---|---|---|
| Result (n = 3) | (hr) | (hr*nmol/L) | (mL/hr/kg) |
| Mean | 105 | 4510 | 0.98 |
| SD | 26 | 1516 | 0.36 |

Abbreviations: AUC_{0-inf} = area under the curve from time 0 hours to infinity; CL = Clearance; T_{1/2} = half-life.

### Oral Administration in Dosage Form Targeting Distal Intestine Release

PK parameters of Compound 2 are determined after a single 20 mg oral dose to male beagle dogs following overnight fasting. Blood samples are collected up to 168 hours post-dose. Plasma is harvested from blood samples by centrifugation and stored frozen (−60 to −80 °C) until analysis using the bioanalytical method described above. Plasma concentrations of Compound 2 are detected through 168 hours post-dose. PK parameters following a single oral dose are presented in Table 19.

TABLE 19
Individual and Mean Pharmacokinetic Parameters Following a Single Oral Dose
of Compound 2 (20 mg) with Sodium Caprate (C10, 280 mg) to Beagle Dogs.

| | | | | | | | | F (AUC, |
|---|---|---|---|---|---|---|---|---|
| | | | | | | | | PO/AUC, |
| | | Dose | T_{1/2} | Tₘₐₓ | Cₘₐₓ | AUC_{0-inf} | CL/F | IV) |
| Formulation | Animal | (nmol/kg) | (hr) | (hr) | (nmol/L) | (hr*nmol/L) | (mL/hr/kg) | (%) |
| Example 3 | Mean | 389 | 96 | 5 | 202 | 16662 | 70.8 | 3.9 |
| | SD | | 10 | 2 | 179 | 13998 | 106.1 | 3.2 |

Abbreviations: AUC_{0-inf} = area under the curve from time 0 hours to infinity; CL/F = apparent clearance; Cₘₐₓ = maximum concentration; Tₘₐₓ = time to maximal concentration; T_{1/2} = half-life; F (AUC, PO/AUC, IV) = absolute bioavailability.

As seen in Table 19, oral administration of Example 3 resulted in mean bioavailability of 3.9% of the bioavailability that was observed when administered Compound 2 was administered subcutaneously.

### Pharmacokinetics of Compound 3 in Beagle Dogs:

### Bioanalytical Summary

Compound 3 and the internal standard are extracted from dog plasma (50 μL) by protein precipitation using methanol. The samples are then centrifuged (3000 rpm for 10 minutes) and the supernatant is transferred to a Lo-Bind plate. The samples are then evaporated and reconstituted in acetonitrile and water (50/50 v/v) containing formic acid (1% v/v). After centrifugation (3000 rpm for 3 minutes), an aliquot (20 μL) is injected on to a 2× Sprite AC1842 Armor C18, 5 μm, 40×2.1 mm column for LC/MS analysis.

### Intravenous Administration

The plasma pharmacokinetics (PK) of Compound 3 are evaluated in male beagle dogs following a single intravenous (IV) dose (0.02 mg/kg or 4.01 nmol/kg). Blood samples are collected over 336 hours. Plasma is harvested from blood samples by centrifugation and stored frozen (−60 to −80 °C) until analysis. Plasma concentrations of Compound 3 are detected according to the bioanalytical method described above. PK parameters after a single IV dose of Compound 3 are presented in Table 20.

TABLE 20
Individual and Mean Pharmacokinetic Parameters Following a Single
IV Dose (0.020 mg/kg or 4.01 nmol/kg) to Beagle Dogs.

| Compound 3 | T_{1/2} | AUC_{0-inf} | CL |
|---|---|---|---|
| N = 3 | (hr) | (hr*nmol/L) | (mL/hr/kg) |
| Mean | 104 | 6626 | 0.61 |
| SD | 6 | 332 | 0.03 |

Abbreviations: AUC_{0-inf} = area under the curve from time 0 hours to infinity; CL = Clearance; T_{1/2} = half-life.

### Oral Administration in Dosage Form Targeting Middle and Distal Intestine Release

PK parameters of Compound 3 are determined after a single 20 mg oral dose of in different formulations to male beagle dogs. Blood samples are collected up to 336 hours post-dose. Plasma is harvested from blood samples by centrifugation and stored frozen (−60 to −80 °C) until analysis using the bioanalytical method described above. Plasma concentrations of are detected through 336 hours post-dose.

PK parameters following a single oral dose of Compound 3 to beagle dogs are presented in Table 21.

TABLE 21
Individual and Mean Pharmacokinetic Parameters Following a Single Oral Dose
of Compound 3 (20 mg) with Sodium Caprate (C10, 280 mg) to Beagle Dogs.

| | | | | | | | F (AUC, |
|---|---|---|---|---|---|---|---|
| | | | | | | | PO/AUC, |
| | | T_{1/2} | Tₘₐₓ | Cₘₐₓ | AUC_{0-inf} | CL/F | IV) |
| Formulation | | (hr) | (hr) | (nmol/L) | (hr*nmol/L) | (mL/hr/kg) | (%) |
| Example 4, | Mean | 111 | 5 | 96.6 | 9476 | 139 | 1.5 |
| System I | SD | 14 | 4 | 108 | 10913 | 137 | 1.8 |
| (n = 5) | | | | | | | |
| Example 4, | Mean | 104 | 4 | 262 | 25558 | 25.3 | 4.1 |
| System II | SD | 11 | 0 | 202 | 23331 | 16.9 | 3.7 |
| (n = 5) | | | | | | | |

Abbreviations: AUC_{0-inf} = area under the curve from time 0 hours to infinity; CL/F = apparent clearance; Cₘₐₓ = maximum concentration; Tₘₐₓ = time to maximal concentration; T_{1/2} = half-life; F (AUC, PO/AUC, IV) = absolute bioavailability.

As seen in Table 21, oral administration of Example 4, System I resulted in mean bioavailability of 1.5% of what was observed when Compound 3 was administered subcutaneously, and oral administration of Example 4, System II resulted in mean bioavailability of 4.10% of what was observed when Compound 3 was administered subcutaneously.

### Pharmacokinetics of Tirzepatide (TZP) in Beagle Dogs

### Bioanalytical Summary

TZP and the internal standard are extracted from dog plasma (50 μL) by protein precipitation using isopropyl alcohol and methanol (50:50 v/v). The samples are then centrifuged (1200 rpm for 5 minutes) and the supernatant is transferred to a Siricco Protein Precipitation Plate. The samples are then loaded on a SepPak tC18 SPE microelution plate that is conditioned with 2% formic acid in acetonitrile followed by 2% formic acid in water. The compounds are then washed with 2% formic acid in water and eluted using 2% formic acid in acetonitrile into a plate containing 1× Invitrosol and 1% formic acid in water prior to injecting an aliquot (10 μL) on to XSelect CSH C18, 3.5 μm, 2.1×30 mm column for LC/MS analysis.

### Intravenous Administration

The plasma pharmacokinetics (PK) of TZP are evaluated in male beagle dogs following a single intravenous (IV) dose (0.02 mg/kg or 4.15 nmol/kg). Blood samples are collected over 336 hours. Plasma is harvested from blood samples by centrifugation and stored frozen (−50 to −100 °C) until analysis. Plasma concentrations of TZP are detected according to the bioanalytical method described above. PK parameters after a single IV dose of TZP are presented in Table 22.

TABLE 22
Individual and Mean Pharmacokinetic Parameters
Following a Single IV Dose of LY3298179 (0.020
mg/kg or 4.15 nmol/kg) to Beagle Dogs.

| | Dose | T_{1/2} | AUC_{0-inf} | CL |
|---|---|---|---|---|
| Route | (nmol/kg) | (hr) | (hr*nmol/L) | (mL/hr/kg) |
| IV | 4.15 | 70 | 7910 | 0.531 |

Abbreviations: AUC_{0-inf} = area under the curve from time 0 hours to infinity; CL = Clearance; T_{1/2} = half-life.

### Subcutaneous Administration

The plasma pharmacokinetics (PK) of TZP are evaluated in male beagle dogs following a single subcutaneous (SC) dose (0.02 mg/kg or 4.15 nmol/kg). Blood samples are collected over 336 hours. Plasma is harvested from blood samples by centrifugation and stored frozen (−50 to −100 °C) until analysis. Plasma concentrations of TZP are detected according to the bioanalytical method described above. PK parameters after a single SC dose of TZP are presented in Table 23.

TABLE 23
Individual and Mean Pharmacokinetic Parameters Following a Single
SC Dose of TZP (0.020 mg/kg or 4.15 nmol/kg) to Beagle Dogs.

| | T_{1/2} | Tₘₐₓ | Cₘₐₓ | AUC_{0-inf} | CL/F |
|---|---|---|---|---|---|
| N = 3 | (hr) | (hr) | (nmol/L) | (hr*nmol/L) | (mL/hr/kg) |
| Mean | 159 | 11 | 24 | 4070 | 1.1 |
| SD | 54 | 2 | 10 | 1030 | 0.3 |

Abbreviations: AUC_{0-inf} = area under the curve from time 0 hours to infinity; CL/F = apparent clearance; Cₘₐₓ = maximal concentration; Tₘₐₓ = time to maximal concentration; T_{1/2} = half-life.

### Oral Administration in Dosage Form Targeting Distal Intestine Release

PK parameters are determined after a single 20 mg oral dose of the dosage form described in Example 8 to male beagle dogs following overnight fasting. Blood samples are collected up to 336 hours post-dose. Plasma is harvested from blood samples by centrifugation and stored frozen (−50 to −100 °C) until analysis using the bioanalytical method described above. Plasma concentrations of TZP are detected through 336 hours post-dose. PK parameters following a single oral dose of TZP to beagle dogs are presented in Table 24.

TABLE 24
Individual and Mean Pharmacokinetic Parameters Following a Single Oral
Dose of TZP (20 mg) with Sodium Caprate (C10, 280 mg) to Beagle Dogs.

| | | | | | | F (AUC, | F (AUC, |
|---|---|---|---|---|---|---|---|
| | | | | | | PO/AUC, | PO/AUC, |
| | T_{1/2} | Tₘₐₓ | Cₘₐₓ | AUC_{0-inf} | CL/F | IV) | SC) |
| N = 8 | (hr) | (hr) | (nmol/L) | (hr*nmol/L) | (mL/hr/kg) | (%) | (%) |
| Mean | 97 | 5 | 119 | 9030 | 124 | 1.4 | 2.6 |
| SD | 28 | 3 | 160 | 12600 | 111 | 1.9 | 3.7 |

Abbreviations: AUC_{0-inf} = area under the curve from time 0 hours to infinity; CL/F = apparent clearance; Cₘₐₓ = maximum concentration; Tₘₐₓ = time to maximal concentration; T_{1/2} = half-life; F (AUC, PO/AUC, IV) = absolute bioavailability; F (AUC, PO/AUC, SC) = relative bioavailability.

As seen in Table 24, oral administration of Example 8 resulted in mean bioavailability of 2.6% of what was observed when tirzepatide was administered subcutaneously.

### Clinical Study

A clinical study is designed to evaluate and compare the PK of different test dosage forms comprising Compound 1 administered as multiple once-daily oral doses in healthy participants with that of a reference tablet formulation. In addition, the PD effects of Compound 1 on body weight, appetite, and food intake will be explored following the 3 consecutive once daily oral doses.

The study is intended to estimate the relative oral bioavailability of various 4-mg oral formulations with 280 mg C10 versus a 4-mg with 300 mg SNAC oral formulation. A population of healthy participants is selected to assess the safety and tolerability of Compound co-formulated with 280 mg C10 for oral administration with an intended lower intestinal release mechanism. A cohort of Compound 1 co-formulated with 300 mg SNAC will be used as the reference cohort. Using a healthy participant population mitigates possible confounding effects of comorbidities and concomitant medications. Therefore, the study provides an unbiased assessment of safety, tolerability, and PK of Compound 1 administered as oral doses. A study design of once-daily oral dosing for 3 days will be employed to characterize oral PK behavior of Compound 1.

The reference tablet formulation will include the permeation enhancer salcaprozate sodium (SNAC), to enable oral absorption of the peptide. Details of the reference composition are provided in Table 25 below:

TABLE 25
Reference tablet formulation.

| Ingredient | Amount (mg) |
|---|---|
| Compound 1 | 4 |
| SNAC | 300 |
| Microcrystalline cellulose | 90 |
| Magnesium stearate | 6 |
| Total Tablet Weight | 400 |

The test capsule formulations include sodium caprate (C10) as a permeation enhancer and are prepared inside SZ0 HPMC capsules. The compositions inside the capsules, referred to herein as the “core compositions,” for all test dosage forms are as shown in Table 26 below:

TABLE 26
Core composition in test dosage forms.

| Component | mg/capsule | w/w % |
|---|---|---|
| Compound 1 | 4.0 | 1.1% |
| Sodium | 280.0 | 80.0% |
| Decanoate | | |
| MCC | 66.0 | 18.9% |
| Total | 350.0 | 100.0% |

Filled capsules are banded using the material shown in Table 27 below.

TABLE 27
Banding material in test dosage forms.

| Component | w/w % |
|---|---|
| HPMC | 17.1 |
| Ethanol | 58.0 |
| Water | 24.9 |

Banded capsules are then coated with different enteric polymer systems at various amount targeting different regions in the gastrointestinal tract. Details of the coating systems and test dosage forms are shown in Tables 28 and 29 below:

TABLE 28
Coating compositions used in test dosage forms.

| | |
|---|---|
| Coating | Coating |
| System I | System II |

| | |
|---|---|
| Component | % w/w |

| | | |
|---|---|---|
| L30D-55 | 64.5 | 12.9 |
| FS30D | — | 51.6 |
| TEC | 2.9 | 2.9 |
| Purified water | 32.6 | 32.6 |

TABLE 29
Coating systems and levels used in test dosage forms.

| Test Dosage | Coating | | |
|---|---|---|---|
| Form | System | Coating amount | |
| 1 | I | 6.5-7.5 | mg/cm² |
| 2 | II | 9.5-10.5 | mg/cm² |
| 3 | II | 6.5-7.5 | mg/cm² |

Objectives and endpoints for the study are set forth in Table 30 below.

TABLE 30
Objectives and Endpoints.

| Objectives | Endpoints |
|---|---|
| Primary | |
| To evaluate and compare the PK of up to 3 | AUC and Cmax |
| test dosage forms with a reference tablet | |
| formulation following 3 consecutive once- | |
| daily oral doses in healthy participants | |
| Secondary | |
| To evaluate the PK of up to two test dosage | AUC and Cmax |
| forms when administered in a fed condition | |
| following 3 consecutive once-daily oral | |
| doses in healthy participants | |

Abbreviations: AUC = area under the concentration versus time curve; Cmax = maximum observed drug concentration; PK = pharmacokinetics.

The study is designed as a 2-part open label randomized study with a parallel design to be conducted in up to 5 cohorts of healthy participants. Part A of the study is an initial characterization of 2 test dosage forms, compared with a reference tablet formulation. Participants will be randomized to 1 of the 3 possible treatments, which will be administered in the fasted state. The 3 cohorts may be conducted in parallel, each cohort targeting 10 participants completing the study—that is, these participants must complete Day 8 PK assessments after receiving all 3 doses of the study treatment.

An initial review of safety and PK data from Part A will be conducted to determine the dosage form(s) to be evaluated in Part B, which will enroll up to 2 cohorts of participants (each targeting 10 to complete Day 8 PK assessments after receiving all 3 doses of the study treatment). Depending on the review results, the selected dosage form(s) will be administered in either the fed or fasted state.

If deemed appropriate, a second review of available PK data from Cohort 4 may be conducted prior to the initiation of Cohort 5. Otherwise, Cohorts 4 and 5 may be initiated concurrently if the formulations “X” and “Y” and administration conditions are determined for both cohorts after the first review.

Participants are eligible to be included in the study only if all of the following criteria apply: are 21 to 65 years of age inclusive, at the time of signing the informed consent; are overtly healthy as determined by medical evaluation including screening medical history, physical examination, vital signs, clinical laboratory tests, and ECG, Have clinical laboratory test results within normal reference range for the population or CRU, or results with acceptable deviations that are judged to be not clinically significant by the investigator; Have venous access sufficient to allow blood sampling as per the protocol; Are able and willing to consume the high-fat, high-calorie breakfast meal provided on dosing days (Part B only); Are reliable and willing to make themselves available for the duration of the study and who will comply with the required study and dosing visits and abide by the clinical research site policy and procedure and study restrictions; have Body mass index (BMI) within the range 19.0 to 40.0 kg/m² (inclusive).

Participants are excluded from the study if any of the following criteria apply: Have a history of atopy (severe or multiple allergic manifestations) or clinically significant multiple or severe drug allergies, or intolerance to topical corticosteroids, or severe posttreatment hypersensitivity reactions (including, but not limited to, erythema multiforme major, linear immunoglobulin A dermatosis, toxic epidermal necrolysis, anaphylaxis, angioedema, or exfoliative dermatitis); Have a significant history of or current CV (for example, myocardial infarction, congestive heart failure, cerebrovascular accident, venous thromboembolism, etc.), respiratory, renal, GI, endocrine, hematological (including history of thrombocytopenia), or neurological disorders capable of significantly altering the absorption, metabolism, or elimination of drugs, of constituting a risk while taking the IP, or of interfering with the interpretation of data; Have a mean supine HR less than 45 bpm or greater than 100 bpm from 2 assessments at screening. If a repeat measurement (mean of 2 assessments) shows values within the range of 45 to 100 bpm, the participant may be included in the trial; Have a mean supine systolic BP higher than 160 mmHg and a mean supine diastolic BP higher than 95 mmHg from 2 assessments at screening; therefore, if a repeat measurement (mean of 2 assessments) shows values within the range (SBP<=160 mmHg and/or DBP<=95 mmHg), the participant can be included in the trial; Have undergone any form of bariatric surgery; Have a history of GI bleeding, or gastric or duodenal ulcers; Have a personal or family history of medullary thyroid carcinoma or have multiple endocrine neoplasia syndrome type 2; Have a history of acute or chronic pancreatitis, or elevation in serum lipase and/or amylase levels greater than 1.5 times the ULN; Have clinical signs or symptoms of liver disease, acute or chronic hepatitis; Have evidence of significant active neuropsychiatric disease as determined by the investigator; Have been treated with prescription drugs that promote weight loss within 3 months prior to screening. Examples include Meridia® (sibutramine), Sanorex® (mazindol), Adipex-P® (phentermine), BELVIQ® (lorcaserin), Mysimba® (naltrexone/bupropion), Saxenda® (liraglutide) or similar other body weight loss medications including any over-the-counter medications or supplements; Have received chronic (lasting >14 consecutive days) systemic glucocorticoid therapy in the past year, or have received any glucocorticoid therapy within 1 month before screening (topical, intra-articular, and inhaled preparations such as steroid nasal spray are permitted in the study); Have an abnormality in the 12-lead ECG at screening that, in the opinion of the investigator, increases the risks associated with participating in the study or may confound ECG (QT) data analysis, such as a QTcF >450 msec for males and >470 msec for females, short PR interval (<120 msec), or PR interval >220 msec, second or third degree atrioventricular block, intraventricular conduction delay with QRS >120 msec, right bundle branch block, left bundle branch block or Wolff-Parkinson-White syndrome; Have serum AST or ALT >1.5 X ULN or TBL >1.5 X ULN; Show evidence of HIV infection and/or positive human HIV antibodies; Show evidence of hepatitis C and/or positive hepatitis C antibody; Show evidence of hepatitis B, positive hepatitis B core antibody, and/or positive hepatitis B surface antigen.

All participants will observe an overnight fast of at least 8 hours prior to the scheduled dosing time and for each subsequent study day when clinical safety laboratory samples are taken. Throughout the inpatient period, apart from the high-fat, high-calorie meal served at dosing, standard meals will be administered in the CRU. While not resident in the CRU, participants will be encouraged to follow their normal diet.

In Cohorts 4 and 5, participants may receive study intervention in the fed state. A standardized high-fat, high-calorie breakfast meal will be served to participants at dosing to achieve a fed state. The standardized high-fat, high-calorie meal should consist of approximately 800-1000 calories. This meal derives approximately 150, 250, and 500-600 calories from protein, carbohydrates, and fat, respectively.

Participants will receive each dose with approximately 240 mL of water. Water ad libitum is allowed only until 1 hour before dosing and following the 2-hour post-dose fast.

Participants will be advised to maintain their regular levels of physical activity/exercise during the study, and to abstain from strenuous exercise for at least 24 hours before each blood collection for clinical laboratory tests. When certain study procedures are in progress at the site, participants may be required to remain recumbent or sitting.

Table 31 below shows the planned study interventions in this study.

TABLE 31
Planned study interventions.

| | | |
|---|---|---|
| ARM Name | Test 1, 2, or 3 | Reference |
| Intervention Name | Test Capsule 1, 2, or 3 | Reference Tablet |
| Type | Drug | Drug |
| Dose Formulation | Capsule | Tablet |
| Unit Dose Strength | 4 mg Compound 1 | 4 mg Compound 1 |
| | with 280 | with 300 |
| | mg C10 | mg SNAC |
| Dosage Level | Single 4-mg dose | Single 4-mg dose |
| Route of | Oral | Oral |
| Administration | | |
| Use | Test formulation | Reference formulation |

All participants observe an overnight fast of at least 8 hours prior to the scheduled dosing time. Water is allowed as desired except as described below and the dosing in the fasted or fed state section. Any additional water required by the participant to complete the dose must be recorded in the CRF.

Compound 1 formulated in one of the test or reference dosage forms is administered orally with room temperature water in the morning of each dosing day in a sitting position. Participants are not allowed to lie supine for 2 hours after dosing, unless clinically indicated or for study procedures.

Participants dosed in the fasted state take each dose on Days 1, 2, and 3 after the overnight fast. After each daily dose, participants continue to fast for 2 hours before receiving a standard meal. Water ad libitum is allowed only until 1 hour before dosing and following the 2-hour post-dose fast.

For participants dosed in the fed state, a high-fat, high-calorie breakfast meal is administered at the same time on each scheduled dosing day, and is consumed within 30 minutes. Participants are administered their assigned treatment 30 minutes after the start of the standard meal on each of the 3 dosing days. Conditions are summarized in Table 32 below.

TABLE 32
Fasting or fed conditions and water consumption for dosing.

| | | | Water for | |
|---|---|---|---|---|
| | | | Dosing | |
| | | Dosing | (approximate | Dosing vs. |
| Cohort | Formulation | Condition | volumes) | Meal Timing |
| 1 | Test 1 | Fasted | 120 mL | Postdose fast |
| | | | | duration: 2 hours |
| 2 | Test 2 | Fasted | 120 mL | Postdose fast |
| | | | | duration: 2 hours |
| 3 | Reference | Fasted | 120 mL | Postdose fast |
| | | | | duration: 2 hours |
| 4 | “X” | TBD^{a} | 120 mL | Postdose fast |
| | | | | duration: 2 hours |
| | | TBD^{a} | 240 mL | Postdose fast |
| | | | | duration: 2 hours |
| | | TBD^{a} | 240 mL | Dosing is scheduled 30 |
| | | | | minutes after |
| | | | | the start of |
| | | | | the breakfast meal |
| 5 | “Y” | Fed | 240 mL | Dosing is scheduled 30 |
| | | | | minutes after |
| | | | | the start of |
| | | | | the breakfast meal |

^{a}The decision to dose under fed or fasted conditions is determined based on review of data from Cohorts 1 to 3.

In Part A of the study, a review of available safety and PK data is conducted to determine the test formulation(s) to be evaluated in Part B. This review includes data from at least 8 participants who have completed 3 consecutive doses of the investigational product in each of the 3 planned Cohorts, and who have completed protocol assessments up to Day 8.

Emerging PK data from Part A determines the formulations tested in Cohort 4 and Cohort 5, as well as the conditions (fed or fasted) under which the test formulations are administered. If deemed appropriate, an interim review of PK data from Cohort 4 is conducted prior to the initiation of Cohort 5. This review includes data from at least 8 participants who have completed 3 consecutive doses of the investigational product, and who have completed protocol assessments up to Day 8.

Plasma samples will be collected for measurement of plasma concentrations of study intervention. The timing of sampling may be altered during the course of the study based on newly available data (e.g. to obtain data closer to the time of peak plasma concentrations) to ensure appropriate monitoring. Concentrations of Compound 1, SNAC, and C10 are assayed using validated liquid chromatography with tandem mass spectrometry methods.

This study evaluates and compares pharmacokinetics of Compound administered as multiple once-daily oral doses in heathy participants. Descriptive statistics, instead of hypothesis testing will be used for evaluating PK and safety.

Pharmacokinetic parameter estimates for Compound 1, C10 and SNAC are calculated using standard noncompartmental methods of analysis. The primary parameters for analysis are Cₘₐₓ, AUC, and tₘₐₓ. Pharmacokinetic parameters for Cₘₐₓ and AUC are computed after the first, second, and third doses. Other parameters, such as half-life, apparent clearance, and apparent volume of distribution, may be reported.

Pharmacokinetic parameters are evaluated to estimate the relative bioavailability. Log-transformed Cₘₐₓ and AUC of Compound 1 from Cohorts 1 to 5 are evaluated in a linear mixed-effects model with a fixed effect for formulation and study day, and a random effect for subject. The estimated ratios of geometric means of different formulations compared to reference (SNAC) and the corresponding 90% CIs are reported. The parameter tₘₐₓ of Compound 1, C10 and SNAC is analyzed nonparametrically using a Wilcoxon rank sum test. Estimates of the median difference and the corresponding 90% CIs are calculated.

1-32. (canceled)

33. A solid oral pharmaceutical dosage form comprising:
a) a core composition comprising a therapeutic protein or peptide and C10;
b) a capsule that contains the core composition and wherein the capsule has a body and a cap;
c) a polymeric seal covering the transition between the capsule cap and body; and
d) an enteric coating that coats the polymeric seal and capsule, wherein the coating comprises one or more co-polymers selected from the group consisting of:
i) poly[methacrylic acid, ethyl acrylate], wherein the methacrylic acid and ethyl acrylate are present in a ratio of about 1:1;
ii) and poly[methyl acrylate, methyl methacrylate, methacrylic acid], wherein the methyl acrylate, methyl methacrylate and methacrylic acid are present in a ratio of about 7:3:1.

34. The dosage form of claim 33, wherein:
the core composition comprises: (i) 5-25 mg of tirzepatide; and (ii) about 280 mg C10;
the polymeric seal comprises: (i) about 17% HPMC; (ii) about 58% ethanol; and
(iii) about 25% water; and
the enteric coating comprises: (i) about 12.9% poly[methacrylic acid, ethyl acrylate], wherein the methacrylic acid and ethyl acrylate are present in a ratio of about 1:1; (ii) about 51.6% poly[methyl acrylate, methyl methacrylate, methacrylic acid], wherein the methyl acrylate, methyl methacrylate and methacrylic acid are present in a ratio of about 7:3:1; (iii) about 2.9% TEC; and (iv) about 32.6% water; and
wherein the coating level is about 10 mg/cm².

35. The dosage form of claim 33 wherein:
the core composition comprises: (i) 1-25 mg of a therapeutic peptide or protein; (ii) about 280 mg C10; and (iii) about 66 mg MCC;
the polymeric seal comprises: (i) about 17% HPMC; (ii) about 58% ethanol; and (iii) about 25% water; and
the enteric coating comprises: (i) about 12.9% poly[methacrylic acid, ethyl acrylate], wherein the methacrylic acid and ethyl acrylate are present in a ratio of about 1:1; (ii) about 51.6% poly[methyl acrylate, methyl methacrylate, methacrylic acid], wherein the methyl acrylate, methyl methacrylate and methacrylic acid are present in a ratio of about 7:3:1; (iii) about 2.9% TEC; and (iv) about 32.6% water;
and wherein the coating level is about 10 mg/cm².

36. The dosage form of claim 33 wherein:
the core composition comprises: (i) 1-25 mg of a therapeutic peptide or protein; (ii) about 280 mg C10; and (iii) about 66 mg MCC;
the polymeric seal comprises: (i) about 17% HPMC; (ii) about 58% ethanol; and (iii) about 25% water; and
the enteric coating comprises: (i) about 64.5% poly[methacrylic acid, ethyl acrylate], wherein the methacrylic acid and ethyl acrylate are present in a ratio of about 1:1; (ii) about 2.9% TEC; and (iii) about 32.6% water;
and wherein the coating level is about 7 mg/cm².

37. The dosage form of claim 33, wherein the relative bioavailability of the therapeutic peptide or protein following oral administration is greater than 1% of what would be achieved through subcutaneous administration.

38. A method of treating a disease or condition in a patient in need thereof comprising administering the dosage form of claim 33.

39. The method of claim 38 wherein the disease or condition is selected from the group consisting of type 2 diabetes mellitus (T2DM), obesity, cardiovascular disease (CVD), non-alcoholic steatohepatitis (NASH), fatty liver disease (FLD), dyslipidemia, metabolic syndrome, cognitive decline, Parkinson's syndrome and Alzheimer's disease.

40. The method of claim 39 wherein the disease or condition is T2DM.

41. The method of claim 39 wherein the disease or condition is obesity.

42. The method of claim 38, wherein the dosage form is administered once daily, twice daily, alternate days, every third day, every fourth day, every fifth day, every sixth day or once weekly.

43. The method of claim 42, wherein the dosage form is administered once daily.

44-53. (canceled)


SEQUENCE LISTING (4 sequences)

SEQ ID NO: 1 - protein, 39 aa, synthetic construct
  REGION 1..39: note = Synthetic Construct
  SITE 2: note = X at position 2 is Aib
  SITE 6: note = X at position 6 is alpha methyl F(2F)
  SITE 10: note = X at position 10 is 4Pal
  SITE 13: note = X at position 13 is alpha methyl L
  SITE 16: note = X at position 16 is Orn
  MOD_RES 17: note = Lys at position 17 is chemically modified by conjugation of the epsilon-amino group of the Lys side chain with(2-[2-(2-amino-ethoxy)-ethoxy]-acetyl)2-(gamma-G lu)-CO-(CH2)16-CO2H
  SITE 20: note = X at position 20 is Aib
  SITE 24: note = X at position 24 is D-Glu
  SITE 25: note = X at position 25 is alpha methyl Y
  MOD_RES 39: note = AMIDATION
YXEGTXTSDXSIXLDXKAQXEFIXXLIEGGPSSGAPPPS

SEQ ID NO: 2 - protein, 39 aa, synthetic construct
  REGION 1..39: note = Synthetic Construct
  SITE 2: note = X at position 2 is Aib
  SITE 6: note = X at position 6 is alpha methyl F(2F)
  SITE 13: note = X at position 13 is alpha methyl L
  SITE 16: note = X at position 16 is Orn
  MOD_RES 17: note = Lys at position 17 is chemically modified by conjugation of the epsilon-amino group of the Lys side chain with(2-[2-(2-amino-ethoxy)-ethoxy]-acetyl)2-(gamma-G lu)-CO-(CH2)16-CO2H
  SITE 20: note = X at position 20 is Aib
  SITE 24: note = X at position 24 is D-Glu
  MOD_RES 39: note = AMIDATION
YXEGTXTSDYSIXLDXKAQXEFIXYLIEGGPSSGAPPPS

SEQ ID NO: 3 - protein, 39 aa, synthetic construct
  REGION 1..39: note = Synthetic Construct
  SITE 2: note = X at position 2 is Aib
  SITE 13: note = X at position 13 is Aib
  MOD_RES 20: note = Lys at position 20 is chemically modified by conjugation of the epsilon-amino group of the Lys side chain with(2-[2-(2-amino-ethoxy)-ethoxy]-acetyl)2-(gamma-G lu)2-CO-(CH2)18-CO2H
  SITE 22: note = X at position 22 is 1-Nal
  MOD_RES 39: note = AMIDATION
YXEGTFTSDYSIXLDKIAQKAXVQWLIAGGPSSGAPPPS

SEQ ID NO: 4 - protein, 39 aa, synthetic construct
  REGION 1..39: note = Synthetic Construct
  SITE 2: note = X at position 2 is Aib
  SITE 13: note = X at position 13 is Aib
  MOD_RES 20: note = Lys at position 20 is chemically modified by conjugation of the epsilon-amino group of the Lys side chain with(2-[2-(2-amino-ethoxy)-ethoxy]-acetyl)2-(gamma-G lu)-CO-(CH2)18-CO2H
YXEGTFTSDYSIXLDKIAQKAFVQWLIAGGPSSGAPPPS